(19) United States
(12) Patent Application Publication
(10) Pub. No.: US 2026/0306394 A1
(43) Pub. Date: Oct. 8, 2026

(54) COSMETIC OR DERMATOLOGICAL GEL PREPARATION

(71) Applicant: BEIERSDORF AG, Hamburg (DE)

(72) Inventor: Dirk SCHARLE-WOLFF, Hamburg (DE)

(21) Appl. No.: 19/484,927

(22) PCT Filed: May 27, 2024
(86) PCT No.: PCT/EP2024/064500

§ 371 Date: Nov. 17, 2025

(30) Foreign Application Priority Data
Jun. 2, 2023 (DE) 10 2023 205 171.0

(51) Int. Cl. A61K 8/92; A61K 8/04; A61K 8/06; A61K 8/31; A61K 8/37; A61Q 9/02

(52) U.S. Cl. CPC A61K 8/922; A61K 8/042; A61K 8/062; A61K 8/31; A61K 8/37; A61Q 9/02; A61K 2800/22; A61K 2800/262; A61K 2800/34; A61K 2800/80

(57) ABSTRACT

A preparation comprising one or more emollients selected from the group consisting of cetearyl isononanoate, decyl oleate, undecane and/or tridecane and up to 10 wt. % lipophilic foaming agents allows provision of a shaving gel that is morphologically dimensionally stable up to 40° C.

The invention is a cosmetic or dermatological gel preparation containing natural emollients, selected from the group consisting of Cetearyl Isononanoate, Decyl Oleate, Undecane and/or Tridecane, and up to 10% by weight of propellant gases. The preparation is free of polyisobutene, acrylate compounds and silicone compounds and is advantageously also free of polyethylene glycols (PEGs). The gel preparation is morphologically dimensionally stable up to 40° C. and in particular enables the provision of a transparent shaving gel.

The provision of sustainable cosmetic products is a mandatory task, with acceptance by the consumer requiring the product properties, such as in particular pleasant sensory properties, optical properties, haptic properties and compatibility, to remain unchanged.

Raw materials and products are sustainable if they predominantly follow the principle that no more may be consumed than can be regrown, regenerated or made available again in the future.

In natural cosmetics, natural raw materials, near-natural raw materials and also nature-identical raw materials are used to provide sustainable products.

“Near-natural” means chemically modified natural raw materials, where the chemical reactions have an example in nature (for example saponification, esterification). To ensure the microbial safety of the product, use can also be made of nature-identical, i.e. fully synthetic, for example preservatives, which represent a third class of natural cosmetic raw materials.

Nature-identical raw materials are synthetically produced, but identical to the naturally occurring raw materials. These in particular include organic acids.

There is no legal definition of the term “sustainable” or “natural cosmetics” either nationally or throughout Europe. However, there are formal criteria for the requirements to be met by such products. They must be produced predominantly or exclusively from natural substances. Natural substances are substances of plant, animal and/or mineral origin, and mixtures and reaction products thereof.

Their provision is also a challenge that arises for shaving products, in particular in gel form. Gel-like formulations are often found on the market. For the consumer, good handling and sensory properties are among the important properties of gel-like products. When creating the formulation, care must therefore be taken to ensure that the viscosity of the formulation is appropriate.

In addition, the spray application of a highly viscous gel is associated with problems. A high viscosity limits the choice of available application options.

The selection of the raw materials that are present in the formulation is of enormous importance both for the rheology of said formulation and for sensory properties, optical properties and haptic properties.

Known shaving products are shaving gels and foams that, admixed with propellant gases, can be applied from a can in gel or foam form.

Shaving foams are usually liquid shaving soaps that are filled into containers together with propellant gases. Surfactants are often added to improve the foaming. Oils or waxes are also added to make the razor blade glide better. Acrylate-based thickeners or silicones are often added to the formulations to improve gliding and thus protect against injuries caused by the razor blade.

While shaving foams swell out of the can immediately ready to use, in the case of post-foaming shaving gels the foam only develops when it is applied to the skin. The gel products are sprayed onto the hand and foam up within a few seconds when spread on the beard area. The strength of the gel is very heavily dependent on external parameters, such as in particular temperature.

It is desirable to provide a sustainable shaving gel that remains dimensionally stable even at relatively high temperatures. It is also desirable to provide a gel that can be dispensed in a morphologically dimensionally stable, gel-like consistency and can only be rubbed to form a foam on the skin in combination with water.

The invention is a cosmetic or dermatological gel preparation comprising one or more emollients selected from the group consisting of Cetearyl Isononanoate, Decyl Oleate, Undecane and/or Tridecane, and up to 10% by weight of lipophilic propellant gases, in particular 2-10% by weight of a propellant gas mixture of isopentane and isobutane, preferably in a weight ratio of 3:1.

The specified proportions relate to the total mass of the respective preparation, where this can also be referred to as an active ingredient solution without propellant gases or as a preparation with propellant gases.

The emollients according to the invention are natural, sustainable raw materials. The preparation is additionally free of polyisobutene, acrylate compounds and silicone compounds and is also advantageously free of polyethylene glycols (PEGs).

According to the invention, the use of polyisobutene, acrylate compounds and silicone compounds and optionally polyethylene glycols (PEGs) is dispensed with. According to the invention, “dispensing with” means that the proportion of these substances in the preparation is less than 0.1% by weight, and so any negligible proportions, for example due to impurities or entrained materials, are still in accordance with the invention.

Preferably, the proportion of these substances is less than 0.01% by weight, in particular less than 0.001% by weight, advantageously 0% by weight, based on the total mass of the preparation.

Surprisingly, it has been found that the shaving gel according to the invention can be provided in transparent form by the use of the emollients according to the invention, in particular Cetearyl Isononanoate. This is an essential property for the consumer, which can now be used for the first time in a sensitive shaving gel. A transparent shaving gel indicates few raw materials and thus a sensitive product and can therefore be used in a variety of ways and in numerous gel products.

A further advantage according to the invention and thus the achievement of the stated object is that the gel according to the invention is still morphologically dimensionally stable at 40° C. and therefore can also be stored at up to 40° C., in contrast to conventional gels which are liquid at these temperatures.

Emollients are agents for making the skin soft and supple (for softening the skin). Emollients are substances that reduce skin roughness and thus make the skin appear softer and smoother. They act by “filling” the spaces between desquamating corneocytes, by increasing cohesion between corneocytes and by “smoothing” the raised edges of individual corneocytes. Examples of commonly used emollients are oils and fats, such as lanolin, mineral oil and Vaseline. Some emollients are, for example, vegetable oils, synthetic and semisynthetic oils, such as cetyl ethylhexanoate, isopropyl myristate, ethylhexyl palmitate, caprylic/capric triglyceride, and some silicones.

According to the invention, however, only natural emollients from the group of Cetearyl Isononanoate, Decyl Oleate, Undecane and/or Tridecane are selected.

Cetearyl Isononanoate, also cetyl stearyl isononanoate, is a wax obtained from plant raw materials. It is a mixture of esters of isononanoic acid (mainly 3,5,5-trimethylhexanoic acid) with cetearyl alcohol.

Cetearyl Isononanoate is also known as uropygial gland oil and is available for example as Cetiol SN® from BASF.

Decyl Oleate is a traditional, medium-spreading emollient for cosmetic applications. It is a clear, slightly yellowish polar oil with a characteristic odor. Decyl Oleate is available for example as Cetiol® V from BASF.

Undecane is naturally found in coconut, palm, prickly pear, gooseberries, algae and fungi.

Tridecane is primarily present in okra (lady's fingers) and other natural sources.

Advantageously, use is made of the mixture of Undecane and Tridecane.

The mixture of Undecane/Tridecane is a nonpolar, clear oil made from 100% renewable raw materials and is available for example as Cetiol® Ultimate from BASF.

Advantageously, the proportion of one or more emollients according to the invention is selected to be 0.1% to 5% by weight, in particular 0.3% to 2% by weight, preferably 0.4% to 1% by weight, in each case based on the total mass of the preparation without propellant gases (active ingredient solution).

The gel preparations according to the invention preferably comprise only natural substances, natural, near-natural and/or nature-identical raw materials. Natural substances are substances of plant, animal and/or mineral origin, and mixtures and reaction products thereof. Substances that are obtained from natural substances only by physical means, are only chemically modified or are produced in a nature-identical manner are to be considered as natural, near-natural and/or nature-identical raw materials, respectively.

Depending on the embodiment, the preparation is understood to be the active ingredient solution without propellant gases or the overall formulation, active ingredient solution and propellant gases.

The preparation according to the invention without propellant gases is advantageously presented as an O/W emulsion. It is liquid during production. The viscosity of the formula is about 1200 mPas (20°) and advantageously has a density of about 1 g/cm³.

Surprisingly, a morphologically dimensionally stable transparent shaving gel is obtained from the liquid preparation according to the invention, the active substance solution, when lipophilic propellant gases are added, in particular when the mixture consisting of isopentane and isobutane is added in a weight ratio of 3:1.

The preparation according to the invention comprising one or more emollients selected from the group consisting of Cetearyl Isononanoate, Decyl Oleate, Undecane and/or Tridecane and up to 10% by weight of lipophilic propellant gases thus enables the provision of a shaving gel that is morphologically dimensionally stable up to 40° C.

Comparative tests were carried out for this purpose.

A prior art shaving gel with polyisobutene and a shaving gel according to the invention with Cetearyl Isononanoate were compared with regard to temperature stability (see Table I).

The known gel already becomes liquid after about an hour at a temperature of above 35° C.

In order to obtain a dimensionally stable shaving gel again, acclimatization of about 12 hours at room temperature must be effected. In the test, the gel structure was examined at 6° C., RT, at 35° C. and 40° C. At 35°, the prior art product was not able to be dispensed as a gel because it was too liquid.

The shaving gel according to the invention with Cetearyl Isononanoate becomes slightly softer at over 35° C., but not liquid. That is to say that application is now possible according to the invention even at relatively high temperatures. In addition, at even higher temperatures, the necessary acclimatization time for the shaving gel to become dimensionally stable again is reduced.

The stability test for both formulas was carried out over a period of 8 months at 6° C., room temperature, 35° C. and 40° C. The gel structures of the formulas were compared and evaluated.

The formula according to the invention had a clear/transparent gel structure at 6° C., RT, >35° C. and 40° C., and a significantly superior durability in the gel structure (test period of 8 months).

TABLE I
Formulation, active ingredient solution

| | | Shaving gel according |
|---|---|---|
| | Prior art shaving gel | to the invention % by |
| INCI | % by weight | weight |
| Panthenol + Aqua + Pantolactone + | 0.01000 | 0.01000 |
| Citric Acid | | |
| Tocopheryl Acetate | 0.01000 | 0.01000 |
| Maltodextrin + Chamomilla Recutita | 0.01000 | 0.01000 |
| Flower Extract | | |
| Isopropyl Palmitate | 1.50000 | 1.50000 |
| Cetearyl Isononanoate | 0 | 0.50000 |
| Oleth-20 | 6.00000 | 6.00000 |
| Palmitic Acid | 8.50000 | 8.50000 |
| Polyisobutene | 0.50000 | 0 |
| Glycerin + Aqua | 2.50000 | 2.50000 |
| Triethanolamine + Aqua | 6.70000 | 6.70000 |
| Piroctone Olamine | 0.05000 | 0.05000 |
| BHT | 0.03000 | 0.03000 |
| Hydroxyethylcellulose + Disodium | 0.40000 | 0.40000 |
| Phosphate + Sodium Phosphate | | |
| Hydroxypropyl Methylcellulose | 0.10000 | 0.10000 |
| PEG-90 Glyceryl | 0.50000 | 0.50000 |
| Isostearate + Aqua + Laureth-2 | | |
| PEG-14M | 0.10000 | 0 |
| Aqua | to 100 | to 100 |

In the shaving gel according to the invention, the propellant gas used is preferably propane, propene, n-butane, isobutane, isobutene, n-pentane, n-pentene, isopentane, isopentene, methane and ethane individually or in combination. Hydrophilic propellant gases, such as carbon dioxide, may also advantageously be used in the context of the present invention if the proportion of hydrophilic gases is selected to be low and lipophilic propellant gas (for example propane/butane) is present in excess. Since the lipophilic propellant gas proportion is then predominant, it is said according to the invention that it is lipophilic propellant gases that are used according to the invention.

The propellant gas proportion is advantageously up to 10% by weight, in particular 2% to 10% by weight, advantageously 3% to 8% by weight, based on the total mass of the preparation including propellant gases.

Isopentane and isobutane are preferably used as propellants. Use is advantageously made of a mixture of isopentane and isobutane with a weight ratio of isopentane to isobutane of 3 to 1.

If only small amounts (<1%) of other propellants, in traces or entrained, are present in the aerosol gel preparation, then the gel preparation is still considered to be provided with only one or the propellant according to the invention.

The demanding cosmetic compositions according to the invention may optionally comprise further customary auxiliaries and additives, such as consistency regulators, fillers, perfume, dyes, active ingredients, vitamins, proteins, light stabilizers, stabilizers, insect repellents, salts, EDTA, antimicrobially, proteolytically or keratolytically active substances etc., provided that they are not excluded according to the invention.

In contrast to known formulations, what has been found according to the invention is a specific combination of a gel formulation and a specific propellant gas which remedies the disadvantages of the prior art.

The emollients according to the invention, selected from the group consisting of Cetearyl Isononanoate, Decyl Oleate, Undecane and/or Tridecane, in combination with up to 10% by weight of lipophilic propellant gases, based on the total mass of the preparation, enable the production of a morphologically dimensionally stable shaving gel up to a temperature of 40° C.

Morphologically, in terms of its external appearance, the shape and structure, the gel according to the invention is dimensionally stable. That is to say that the gel is spongy, no foam, but stable in its gel form and can easily be spread on the skin.

The examples below elucidate the active substance solutions according to the invention. The figures are percentages by weight based on the total weight of the preparation without propellant gases.

| | 1% | 2% |
|---|---|---|
| Examples: INCI | by weight | by weight |
| Undecane/Tridecane (Cetiol | 0 | 0.50000 |
| Ultimate) | | |
| Panthenol + Aqua + Pantolactone + Citric | 0.01000 | 0.01000 |
| Acid | | |
| Tocopheryl Acetate | 0.01000 | 0.01000 |
| Maltodextrin + Chamomilla Recutita | 0.01000 | 0.01000 |
| Flower Extract | | |
| Isopropyl Palmitate | 1.50000 | 1.50000 |
| Decyl Oleate + Tocopherol | 0.50000 | 0 |
| Oleth-20 | 6.00000 | 6.00000 |
| Palmitic Acid | 8.50000 | 8.50000 |
| Polyisobutene | 0 | 0 |
| Glycerin + Aqua | 2.50000 | 2.50000 |
| Triethanolamine + Aqua | 6.70000 | 6.70000 |
| Piroctone Olamine | 0.05000 | 0.05000 |
| Aqua | 73.19000 | 73.19000 |
| BHT | 0.03000 | 0.03000 |
| Hydroxyethylcellulose + Disodium | 0.40000 | 0.40000 |
| Phosphate + Sodium Phosphate | | |
| Hydroxypropyl Methylcellulose | 0.10000 | 0.10000 |
| PEG-90 Glyceryl | 0.50000 | 0.50000 |
| Isostearate + Aqua + Laureth-2 | | |

The preparations are then admixed with propellant gases in suitable application means. The proportion of propellant gases based on the total mass of the preparation including the propellant gases is advantageously 2% to 10% by weight, and it is possible to dispense dimensionally stable gels even at high temperatures (up to 40° C.).

1.-9. (canceled)

10. A cosmetic or dermatological gel preparation, wherein the preparation comprises (i) one or more emollients selected from Cetearyl Isononanoate, Decyl Oleate, Undecane and Tridecane and (ii) up to 10% by weight of one or more lipophilic propellant gases, based on a total weight of the preparation, and is free of polyisobutene, acrylate compounds and silicone compounds.

11. The preparation of claim 10, wherein the preparation comprises Cetearyl Isononanoate.

12. The preparation of claim 10, wherein the preparation comprises from 0.1% to 5% by weight of (i), based on a total weight of the preparation.

13. The preparation of claim 12, wherein the preparation comprises from 0.3% to 2% by weight of (i).

14. The preparation of claim 12, wherein the preparation comprises from 0.4% to 1% by weight of (i).

15. The preparation of claim 10, wherein (ii) comprises one or both of isobutane and isopentane.

16. The preparation of claim 10, wherein (ii) comprises isobutane and isopentane in a weight ratio of 1:3.

17. The preparation of claim 10, wherein the preparation comprises from 2% to 10% by weight of a mixture of isopentane and isobutane.

18. The preparation of claim 17, wherein isobutane and isopentane are present in a weight ratio of 1:3.

19. The preparation of claim 10, wherein the preparation is free of polyethylene glycols (PEGs).

20. The preparation of claim 10, wherein apart from (ii) only natural, near-natural and/or nature-identical raw materials are present.

21. The preparation of claim 10, wherein the preparation is present as a shaving gel.

22. The preparation of claim 21, wherein the shaving gel is morphologically stable at a temperature of 40° C.

23. The preparation of claim 21, wherein the shaving gel can be dispensed in a morphologically stable gel-like consistency and can only be rubbed to form a foam on skin in combination with water.

24. The preparation of claim 21, wherein the shaving gel is transparent.

25. The preparation of claim 20, wherein the preparation without (ii) is an O/W emulsion.

26. The preparation of claim 10, wherein the preparation has a viscosity at 20° C. of about 1200 mPas.

27. A method of making a gel which is morphologically dimensionally stable at a temperature of 40° C., wherein the method comprises incorporating in a gel preparation one or more of Cetearyl Isononanoate, Decyl Oleate, Undecane and Tridecane and up to 10% by weight of lipophilic propellant gases, based on a total weight of the preparation.
